(19) United States
(12) Patent Application Publication
(10) Pub. No.: US 2017/0112737 A1
(43) Pub. Date: Apr. 27, 2017

(54) COSMETICS AND PHARMACEUTICAL APPLICATIONS OF GALLIC ACID AND GALLIC ACID DERIVATIVES

(71) Applicants: GREENPHARMA, Orleans (FR); BIOALTERNATIVES, Gencay (FR)

(72) Inventors: Philippe BERNARD, La Ferté Saint Aubin (FR); François-Xavier BERNARD, Saint Maurice La Clouere (FR); Franck HIMBERT, Orléans (FR)

(21) Appl. No.: 15/127,102

(22) PCT Filed: Mar. 18, 2015
(86) PCT No.: PCT/FR2015/050664

§ 371 Date: Sep. 19, 2016

(30) Foreign Application Priority Data
Mar. 18, 2014 (FR) 14/52205
Jan. 14, 2015 (FR) 15/50295

(51) Int. Cl. A61K 8/368; A61K 8/97; A61K 45/06; A61Q 17/00; A61K 31/194; A61Q 19/00; A61Q 19/08

(52) U.S. Cl. CPC A61K 8/368; A61Q 19/00; A61K 8/97; A61Q 19/08; A61Q 19/007; A61Q 17/00; A61K 31/194; A61K 45/06

(57) ABSTRACT

The present invention concerns a compound, a salt of this compound, a derivative of this compound or a salt of this derivative, said compound being chosen among the gallic acid, its hydrolyzable polymers in gallic acid, the hexahydroxydiphenic acid, its hydrolyzable polymers in hexahydroxydiphenic acid, the ellagic acid, its hydrolyzable polymers in ellagic acid, the gallotannins, the ellagitannins, and its cosmetic applications for stimulating or repairing the barrier function of the epidermis and its pharmaceutical applications for treating lesions caused by pathologies, such as Crohn's disease.

### TECHNICAL FIELD

The present invention concerns a compound, its salts and its derivatives, said compound being chosen among the gallic acid, the hexahydroxydiphenic acid, the ellagic acid and the derivatives of these acids and in particular the gallotannins and the ellagitannins, as well as its cosmetic and pharmaceutical applications.

### BRIEF SUMMARY

Indeed, the authors of the invention have discovered that the compounds of the invention are effective in restoring the barrier function of human or animal organs, whether they are external such as the epidermis or internal such as those of the digestive tract, thereby allowing reinforcing the protection of the organs against the aggressions they are subjected to, for example against environmental aggressions regarding the epidermis or lesions associated to a pathological state. Such properties are particularly interesting for preventing or treating affections of the skin such as acne, atopic dermatitis, psoriasis, eczema, dryness of the skin with an atopic tendency, rednesses, but also for preserving young skins, as well as aged skins, from natural or premature ageing or for preventing or treating pathologies of an internal organ or the consequences of these pathologies, such as Crohn's disease.

The authors of the invention have demonstrated that a compound hereinabove allows stimulating the expression of different protein markers essential in the formation of the stratum corneum or horny layer, including the key enzyme, the transglutaminase TGK.

The stratum corneum is the most superficial portion of the skin and represents the culmination of the differentiation process of the keratinocytes. During this differentiation, the keratinocytes originating from the basal underlayer undergo a series of metabolic and structural reorganizations all along their migration towards the surface of the skin. The last stage is their transformation into corneocytes whose accumulation into a cohesive structure constitutes the stratum corneum which ensures the barrier function. All along the differentiation process, several proteins intervene.

One of them, the TGK, of the glutaminases family catalyzes the establishment of covalent peptidic bonds of the ε(γ-glutamyl)-lysine type between various protein precursors which form the stratum corneum and which confer to the latter this capability of protecting the skin from external aggressions and limiting the diffusion of water coming from the inside. The transglutaminase forms generally insoluble polymers of proteins. These biological polymers are indispensable for the organism for creating barriers and stable structures.

The present invention specifically aims to offer new compositions capable of stimulating the factors intervening in the differentiation, called the pro-differentiation factors such as the TGK, thereby allowing improving the conditions of the skin, appendages, as well as mucosae, whether healthy or affected, and therefore reinforcing their functions, but also those of the wall of internal organs.

The Crohn's disease is a chronic inflammatory disease which may affect the entire digestive tract. Most often, it develops in the intestines resulting in an alteration of the wall by ulcerations and tears of the wall resulting in the loss of its barrier function.

Dermatitises are affections of the skin and of the mucosae, which are characterized by unsightly manifestations such as rednesses and desquamation peels. Several pathologies are grouped under the denomination of dermatitises. As non-limiting examples, mention may be made to eczema, atopic dermatitis, psoriasis, seborrheic dermatitises or still acne. Dermatitises result in alterations of the barrier function of the epidermis reflected by a permeability of the skin, a cutaneous dryness and a general alteration of the protective functions of the skin against the external environment.

Ageing, whether natural or induced for example by exposure to the sun, or by medicinal treatments, also results in this cutaneous morphological type with an alteration of the barrier function of the epidermis.

Thus, the invention concerns the applications of a compound, of a salt of this compound, of a derivative of this compound or of a salt of this derivative, said compound being chosen among the gallic acid, its hydrolyzable polymers in gallic acid, the hexahydroxydiphenic acid, its hydrolyzable polymers in hexahydroxydiphenic acid, the ellagic acid, its hydrolyzable polymers in ellagic acid, the gallotannins and the ellagitannins.

Among the gallotannins, mention may be made, in a non-limiting manner, to the tannic acid and the 1,2,3,4,6-penta-O-galloyl-beta-D-glucopyranose. Among the ellagitannins, mention may be made, though without restriction, to vescalagin, castalagin, casuarinin, stachyurin, salicarinin A (vescalagin-stachyurin dimer), salicarinin B (vescalagin-casuarinin dimer), and salicarinin C (castalagin-casuarinin dimer).

The structure of these compounds is reproduced hereinafter:

The gallic acid:

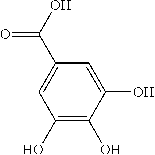 [SMILES: O=C(O)c1cc(O)c(O)c(O)c1]

The hexahydroxydiphenic acid (HHDP):

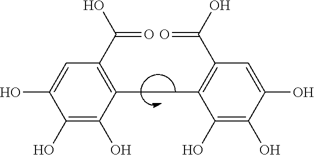 [SMILES: O=C(O)c1cc(O)c(O)c(O)c1Cc1c(C(=O)O)cc(O)c(O)c1O]

The ellagic acid:

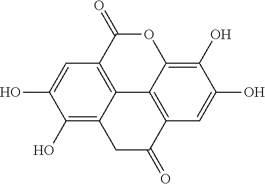 [SMILES: O=C1Cc2c(O)c(O)cc3c(=O)oc4c(O)c(O)cc1c4c23]

The 1,2,3,4,6-penta-O-galloyl-beta-D-glucopyranose:

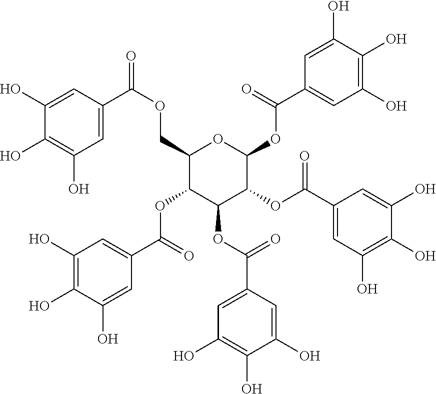 [SMILES: O=C(OC[C@H]1O[C@@H](OC(=O)c2cc(O)c(O)c(O)c2)[C@H](OC(=O)c2cc(O)c(O)c(O)c2)[C@@H](OC(=O)c2cc(O)c(O)c(O)c2)[C@@H]1OC(=O)c1cc(O)c(O)c(O)c1)c1cc(O)c(O)c(O)c1]

Vescalagin:

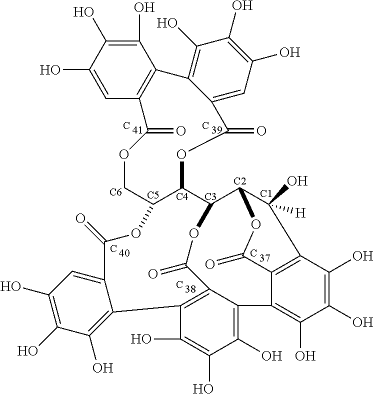

Castalagin:

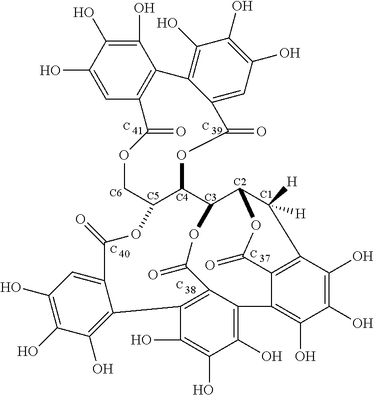

Casuarinin:

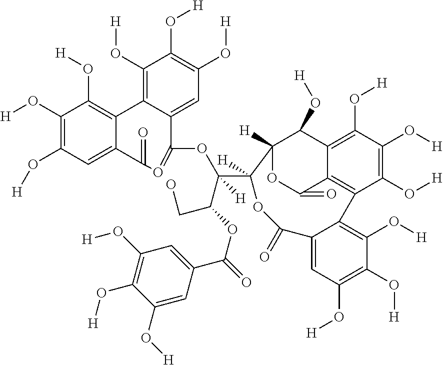

Stachyurin:

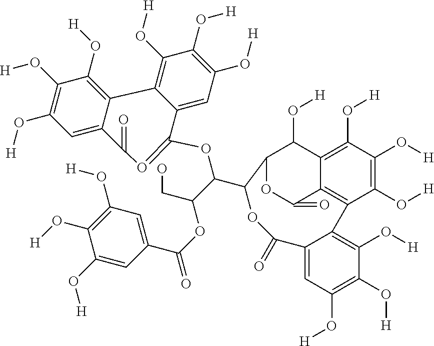

Salicarinin A (Vescalagin-Stachyurin Dimer):

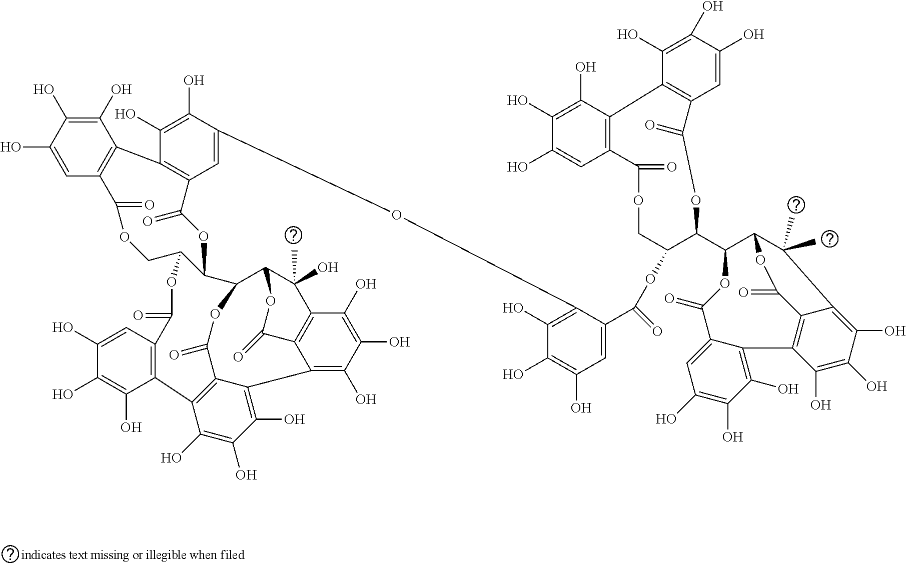

Salicarinin B (Vescalagin-Casuarinin Dimer):

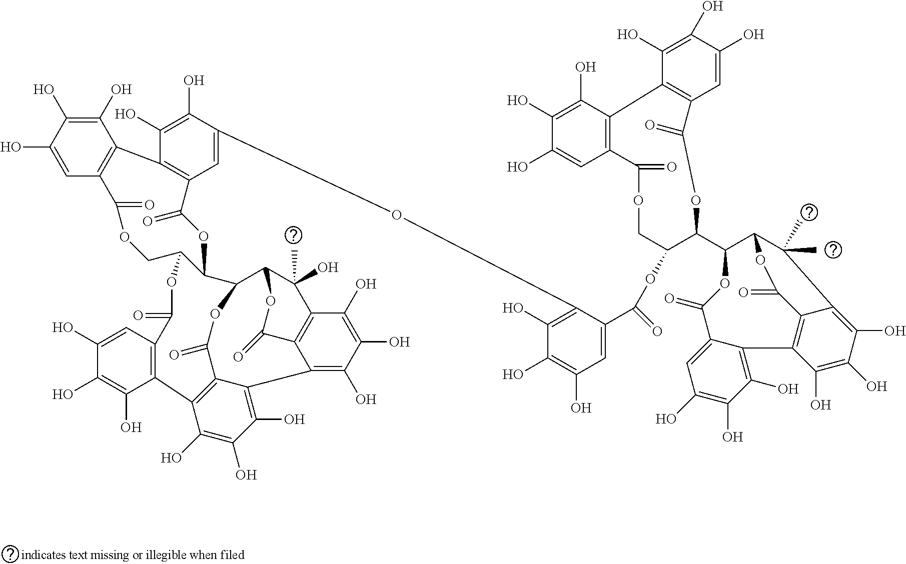

Salicarinin C (Castalagin-Casuarinin Dimer):

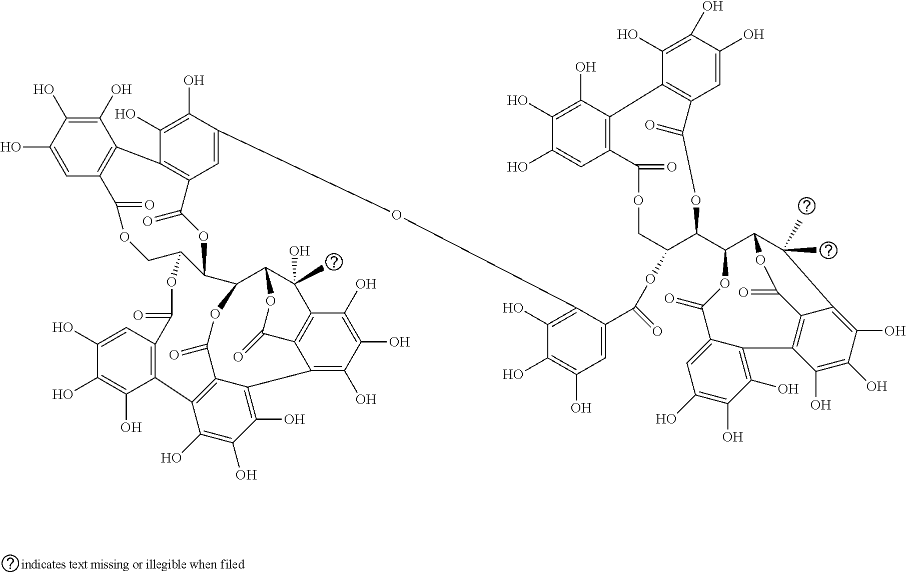

Advantageously, a derivative of said compound has a structure which comprises at least one remainder of the hexahydroxydiphenic acid (HHDP) and a saturated aliphatic hydrocarbon chain of 6 atoms of carbon, said remainder of HHDP being attached on said chain by its carboxyl groups. Said hydrocarbon chain may possibly be substituted with a group chosen among the alkyl groups in C1-C6 and the cycloalkyl groups in C3-C6, possibly interrupted by at least one or several atom(s) of oxygen and possibly substituted with one or several group(s) chosen among the alkyl groups in C1-C6, the cycloalkyl groups in C3-C6, the hydroxyl group and the alkoxyl groups in C1-C6.

A compound hereinabove may have one or several asymmetric center(s). According to the invention, the compound may be in an optically-active form or in the form of its racemic mixture.

In the definition of a derivative hereinabove, the term <<alkyl>> refers to a linear or branched monovalent hydrocarbon radical having advantageously from 1 to 6 atoms of carbon, such as the methyl, ethyl, propyl, isopropyl, butyl, isobutyl, tert-butyl, pentyl, neopentyl, n-hexyl. The alkyl groups may be substituted with one or several hydroxyl group(s) and/or with one or several alkoxy group(s). The term <<cycloalkyl>> a refers to a cyclic monovalent hydrocarbon radical, comprising from 3 to 6 atoms of carbon and being possibly mono- or poly-cyclic. Mention may be made in particular to the <<cyclopropyl>> and cyclohexyl radicals. The <<alkoxy>> groups correspond to the linear or branched alkyl groups defined hereinabove linked via an —O-(ether) bond. Quite particularly, the methoxy, ethoxy, n-propyloxy, i-propyloxy, n-butoxy, s-butoxy, t-butoxy, n-pentoxy and s-pentoxy groups are preferred. The alkoxy groups may be substituted with an alkyl group as defined hereinabove or with another alkoxy group.

By hydrolyzable polymers in gallic acid and hydrolyzable polymers in hexahydroxydiphenic acid, are meant in particular polymers which are partially or completely hydrolyzed, by the skin at its surface or in any one of its layers, taking into consideration that the gallic acid and the hexahydroxydiphenic acid, as well as said partially hydrolyzed polymers are active.

Some compounds of the invention may be obtained from plants, by extraction and are therefore easily available. They may also be used according to the present invention in the form of extracts of plants.

Of course, according to the invention, a compound may be obtained by any process in particular by an organic synthesis, or by isolation from a vegetable source and in particular from a plant, such as a plant from the Lythraceae family, the Fagaceae family, the Myrtaceae family, the Combretaceae family, the Elaeagnaceae family, the Melastomataceae family, the Myricaceae family or the Rosaceae family. For example, the following species of plants belong to these families: Lythrum salicaria L., Quercus sp, Castanea sp, Anogeissus leiocarpus (DC.) Guill. &Perr., Castanea sativa Mill., Corymbia citriodora, Elaeagnus rhamnoides (L.) A. Nelson, Fragaria chiloensis (L.) P. Mill., Fragaria vesca L., Lagerstroemia speciosa (L.) Pers., Melastoma candidum D. Don, Psidium guajava L., Punica granatum L., Quercus suber, Quisqualis indica, Rhodomyrtus tomentosa and Terminalia arjuna. They are particularly interesting as they are widely available and contain quite considerable amounts of one or several of the compounds of the invention.

According to the invention, the compound may be in a salified form, thus it covers every salt of said compound. Advantageously, this salt is cosmetically- or pharmaceutically-acceptable. A salt of a compound of the invention may be an acid-addition salt, said acid being preferably chosen among the hydrochloric acid, the hydrobromic acid, the sulfuric acid, the phosphoric acid, the acetic acid, the trifluoroacetic acid, the lactic acid, the pyruvic acid, the malonic acid, the succinic acid, the glutaric acid, the fumaric acid, the tartaric acid, the maleic acid, the citric acid, the ascorbic acid, the methane- or ethane-sulfonic acid, and the camphoric acid. It may also be a base-addition salt, the latter being preferably chosen among the sodium or potassium hydroxide, the trimethylamine or the tert-butylamine.

The invention concerns the cosmetic applications of a compound or of salt of this compound, as defined hereinabove, but also the mixture of compounds and/or salts and/or derivatives thus defined. Hence, the invention concerns their uses for stimulating or repairing the barrier function of the epidermis. More particularly, it focuses on their uses as an active ingredient in a cosmetic composition for stimulating or repairing the barrier function of the epidermis.

In this regard, the concentration of said compound, salt or derivative or of their mixtures varies from 0.001% to 5% by weight relative to the total weight of the composition, preferably from 0.01% to 5%, still from 0.05% to 5%, by weight relative to the total weight of the composition.

In addition, the invention relates to the pharmaceutical applications, including the veterinary ones, of a compound, salt or derivative as defined hereinabove. Thus, it is of particular interest when it is used in the prevention or the treatment of lesions caused in the walls of organs by pathologies, such as Crohn's disease, in particular in the intestinal wall. Advantageously, it is also intended for the prevention or the treatment of lesions associated to dermatitises such as the atopic dermatitis, eczema, psoriasis, seborrheic dermatitises and acne.

It also concerns a pharmaceutical composition intended to prevent or treat the lesions caused by pathologies, such as Crohn's disease and containing an effective amount of a compound of the invention, or of a salt of this compound, of a derivative of this compound or of a salt of this derivative, as previously defined, and a pharmaceutically-acceptable carrier.

Said salt is compatible with a pharmaceutical use and is preferably an addition salt of the hydrochloric acid, the hydrobromic acid, the sulfuric acid, the phosphoric acid, the acetic acid, the trifluoroacetic acid, the lactic acid, the pyruvic acid, the malonic acid, the succinic acid, the glutaric acid, the fumaric acid, the tartaric acid, the maleic acid, the citric acid, the ascorbic acid, the methane- or ethane-sulfonic acid, or the camphoric acid, or an addition salt of bases chosen among the sodium or potassium hydroxide, the trimethylamine or the tert-butylamine.

In this specific indication, the compound, its salt or the composition comprising it/them, is intended for oral administration.

Advantageously, a pharmaceutical composition of the invention further has the characteristics hereinafter, considered alone or in combination.
the compound is in the form of a powder, in a form supported by absorption on a powdered organic polymer such as bentonite or talc, or in an encapsulated form;
the compound is encapsulated in microspheres, liposomes, glycospheres, chylomicrons, macro-, micro-, or nanoparticles, or macro-, micro-, or nanocapsules;
the composition contains an active substance effective in chronic inflammations, such as an active substance chosen among mucopolysaccharides, vitamins, ceramides and vegetable oils;
the composition includes at least one active agent other than a compound of the invention intended to exert at least a complementary or synergistic action.

According to one aspect of the invention, regardless of the cosmetic or pharmaceutical, including veterinary, applications, the compound, its salt or derivative and their mixtures are used in the form of an extract of a plant, without a complete purification.

By <<extract of a plant>>, is meant an extract or a mixture of extracts of plants from the families listed before. More specifically, it consists of an extract or a mixture of extracts of cells of these families. This cellular material may be obtained by culture in vitro or in vivo. By culture in vitro, are meant all the techniques known by those skilled in the art which allow obtaining a vegetable or a portion of a vegetable in an artificial manner. Thus, the extract may be an extract or a mixture of extracts of an organ (root, stem, leaf, bark, flower), or still of cells of an organ, of at least one plant from the aforementioned families, or still further an extract of undifferentiated cells of at least one such plant.

An extract according to the invention may be obtained by any extraction or purification method known by those skilled in the art. In particular, mention may be made to solid-liquid extraction methods in alcoholic (in particular methanolic, ethanolic) aqueous media, as well as in media using solvents such as ketones, esters, ethers, polyols, chlorinated solvents and the mixtures of at least two of the aforementioned solvents, such as hydroalcoholic media.

Advantageously, an extract is obtained by extraction from one of the aforementioned families of plants in a hydroalcoholic medium, in particular an aqueous solution of ethanol. According to this variant, the concentration of ethanol preferably varies from about 20 to about 40% (v/v), still better it is about 30% (v/v).

Methods other than solvents may also be considered such as supercritical CO₂, microwaves . . . .

These preparation modes form an integral part of the invention.

These extracts may be used as such in a liquid or powdered form, whether purified or not.

If the extract is a powder, drying it may be carried out by any technique well known by those skilled in the art. As example, the extract may be dried by spray-drying, evaporation or lyophilization. The thus obtained powder may be encapsulated in liposomes or other vectors and supports for a better homogeneity of the composition and a better diffusion of the active substance, in particular on the skin.

When the used plant according to the invention is loosestrife, the extract is preferably obtained from flowering tops. The authors have observed that the extracts obtained by hydroalcoholic maceration of the aerial portions of loosestrife present a high biological activity, regardless of the percentage of ethanol in the extraction solvent. Similar results have been observed in water or in ethanol.

The applications according to the invention cover the treatment of appendages and mucosae. By appendages according to the invention, are included, in particular, the nails and the hair system, in particular the hair. By mucosae, are meant the coating tissues of the anatomical cavities, which join the skin by natural orifices, such as the mouth, the stomach, the intestine, as well as those of the external natural cavities such as the nostrils, the ears.

Thus, the present invention also relates to the cosmetic use of a compound or of a salt of said compound, or of a mixture of said compounds and/or of their salts, for protecting the skin, the appendages and the mucosae from physical, chemical or biological external agents, and/or for improving and/or reinforcing the hydration of the skin and/or reinforcing the appendages and the mucosae.

It also concerns a non-therapeutical cosmetic treatment method, for fighting skin dryness or for the cosmetic treatment of a dry skin, said method comprising a step according to which at least one compound, salt or derivative, or their mixtures as previously defined, is applied on the skin.

The invention also relates to a non-therapeutical cosmetic method of anti-ageing treatment of the skin or of non-therapeutical cosmetic treatment of an aged skin comprising a step according to which at least one compound, salt or derivative, or their mixtures as previously defined, whether synthetic or extracted from a plant, is applied on the skin. This treatment allows delaying the skin ageing phenomena, but also repairing an aged skin.

In every cosmetic application of the invention, a compound, salt or derivative, or their mixtures as previously defined, may be associated to another or to other skin pro-differentiation agent(s), such as calcium, vitamin D and their derivatives.

According to another aspect of the invention, a composition according to the invention is in the form of capsules, cream, gel, lotion, milk, oil-in-water or water-in-oil emulsion, solution, ointment, body oil, shampoo, soap, protective lipstick, makeup stick and pencil.

When prepared in the form of a gel, the composition may comprise adequate excipients, such as cellulose esters, or other gelling agents, such as the carboxylic polymer <<Carbopol®>>, and the guar gum, for example.

When prepared in the form of emulsions, the compositions according to the invention present a good stability and may be preserved during the time necessary for their use at temperatures comprised between 0 and 50° C., with no sedimentation of the constituents or separation of the phases.

According to another aspect of the invention, in the composition, a compound, salt or derivative, or their mixtures as previously defined, is set in place in encapsulation means chosen in the group formed by microspheres, liposomes, glycospheres, chylomicrons, macro-, micro- and nanoparticles, macro-, micro- and nanocapsules.

According to another aspect of the invention, in the composition, a compound, salt or derivative, or their mixtures as previously defined, is absorbed or adsorbed on powdery organic polymers, talcs, bentonite or other powdered mineral supports well known by those skilled in the art.

According to another aspect of the invention, in the composition, a compound, salt or derivative, or their mixtures as previously defined, constitutes from 0.001% to 5% by weight relative to the total weight of the composition, preferably from 0.01% to 5%, or still from 0.05% to 5%.

According to another aspect of the invention, in the composition, a compound, salt or derivative, or their mixtures as previously defined, is in an encapsulated form and constitutes, preferably from 0.05 to 5% by weight relative to the total weight of the composition.

Thus, an object of the present invention is a cosmetic use of at least one compound, salt or derivative, or their mixtures as previously defined, for relieving dry skins with an atopic tendency and/or for improving and/or reinforcing the hydration of the skin. In particular, it concerns the uses of at least one compound, salt or derivative, or their mixtures as previously defined, as an active ingredient in a cosmetic composition intended to be applied on a dry skin with an atopic tendency, for fighting skin ageing such as ageing related to the age and/or to photo-ageing, for fighting itches and pruritus, for hydration and for protection from external aggressions related to pollution and stress.

In the therapeutical use of the invention, a compound, salt or derivative, or their mixtures as previously defined, may be administered by topical application but it may also be administered orally. It may be used as such in a liquid or powdered form, whether purified or not.

A compound, salt or derivative, or their mixtures as previously defined, may be associated, in the compositions according to the invention, to other compounds completing the effect or still synergizing this effect.

Its protective activity against free radicals and UVs is interesting in the capillary field, in particular when associated with substances enhancing the well-being of the scalp and of the hair. Mention may be made to the associations with mucopolysaccharides, minerals, vitamins, ceramides, vegetable oils, antiradical substances, U.V. filters, acids of flowers or fruits.

Similarly, the repairing activity of a compound, salt or derivative, or their mixtures as previously defined, is particularly interesting, when associated with substances having a scarring effect such as proteins, the hyaluronic acid, amino acids, or with anti-inflammatory, anti-ageing, after-sun, anti-acne or anti-dermatitis substances.

Thus, the compositions of the invention are quite particularly suitable to a topical application for preventing and/or treating numerous cutaneous alterations, in particular as a repair and/or protective agent of the skin and of the hair system such as the hair, for fighting external aggressions related to pollution, to the sun, to oxidative stress, to ageing and to cutaneous pathologies resulting in a dysfunction of the homeostasis of the epidermis or of the hair.

These cosmetic compositions may also be in the form of a lotion or solution in which the derivatives according to the invention are in an encapsulated form, for example in microspheres. For example, these microspheres may be constituted by fatty bodies, agar and water. The active agents may also be incorporated in vectors such as liposomes, glycospheres, in chylomicrons, macro-, micro-, nanoparticles, as well as macro-, micro- and nanocapsules, and they may also be absorbed on powdered organic polymers, talcs, bentonites and other mineral supports.

In order to prepare the compositions according to the invention, a compound, salt or derivative, or their mixtures as previously defined, may be mixed with excipients generally employed in cosmetics. Hence, the cosmetic compositions of the invention may contain additives or adjuvants commonly-used in cosmetics, such as for example antibacterial agents or perfumes but also extracted and/or synthetic lipids, gelling and viscosifying polymers, surfactants, emulsifiers, hydro- or lipo-soluble active substances, extracts of plants, tissular extracts, marine extracts, or synthetic active substances.

A dermocosmetic or pharmaceutical composition of the invention comprises all body and skin care products, including protective and tanning solar products, anti-ageing products, anti-seborrheic products, tonic products, products intended for the improvement of the aspect of the skin including acne treatment, treatment of cutaneous rednesses, treatment of the scrap and treatment of hair loss.

The cosmetic compositions of the present invention may also comprise other complementary active agents chosen for their action, for example for solar protection, for anti-wrinkle activity, for antiradical and antioxidant activity, for anti-irritant activity, for cell nutrition, for cell respiration, for hydration and for cell regeneration, for anti-seborrheic treatments, as well as other active agents having an action on the cutaneous tonicity and the protection of the hair.

Preferably, the cosmetic compositions of the present invention are to be used on a daily basis by applying them once or several times a day.

The cosmetic compositions of the present invention are very well tolerated, they do not present any phototoxicity and their application on the skin, for long periods of time, does not imply any systemic effect.

The invention also concerns the use of a compound, of a salt of this compound, of a derivative of this compound, of a salt of this derivative, or of their mixtures as previously defined, for the preparation of pharmaceutical compositions presenting an anti-inflammatory and/or dermoprotective activity. These compositions are useful in particular for preventing and/or treating dermatological diseases related to seborrheic, acne, inflammatory and immunological activities.

### BRIEF DESCRIPTION OF THE DRAWINGS

The present invention is now illustrated by the examples given hereinafter. They refer to FIGS. 1 to 11, according to which:

FIG. 1 represents the chromatogram of the loosestrife extract obtained by semi-preparative HPLC under the conditions described in Example 2, representing the concentration (expressed in absorbance units) of the constituents of a loosestrife extract as a function of the retention time (in minutes).

FIGS. 2, 3 and 4 represent the effects of a loosestrife extract and of vescalagin on the release of three mediators, MMP-1 (FIG. 2). MMP-3 (FIG. 3) and procollagen 1 (FIG. 4) by reconstructed skins after 9 days in a depleted medium.

FIG. 5 represents the influence of a loosestrife extract, EX10MF1046, on the expression of different markers of the epidermal differentiation, by the cultures of keratinocytes in the basal state, evaluated by RT-qPCR.

FIG. 6 presents the digital images of sections of reconstructed epidermises (RHE) treated by a control test, a reference and the loosestrife extract, EX10MF1046, at 20 μg/ml, and their effects on the expression of markers of the epidermal differentiation.

FIG. 7 represents the influence of the loosestrife extract, EX10MF1046, on the expression of different markers of the epidermal differentiation, by the cultures of keratinocytes stimulated, or not, by a mixture of cytokines IL-17, OSM and TNFα (mix-PSO), evaluated by RT-qPCR

FIG. 8 presents the digital images of sections of reconstructed epidermises stimulated by a mixture of cytokines IL-17, OSM, TNFα treated by a control test, a reference (JAK inhibitor 1) and the loosestrife extract, EX10MF1046 at 20 μg/ml, and their effects on the expression of the epidermal differentiation marker KRT10.

FIG. 9 represents the influence of the loosestrife extract, EX10MF1046, on the expression of different chemokines induced by the inflammation, by the cultures of keratinocytes stimulated, or not, by a mixture of cytokines IL-17, OSM and TNFα (mix-PSO), evaluated by RT-qPCR.

FIG. 10 represents the influence of the loosestrife extract, EX10MF1046, on the release of the IL-8 by reconstructed epidermises (RHE on D13) stimulated by the mixture of cytokines IL-17, OSM and TNFα (mix-PSO) for 48 h, observed in FIG. 7.

FIG. 11 presents a scheme of a chronic inflammation loop of the skin (for example, psoriasis) illustrating the stage at which the loosestrife extract, EX10MF1046, intervenes in this loop.

### EXAMPLES

### Example 1: Obtaining Loosestrife Extracts (Lythrum salicaria L) Comprising a Compound of the Invention

Different loosestrife extracts (EX3MF1046, EX4MF1046, EX1MF1046 and EX2MF1046) have been obtained in the present example, according to a general protocol described hereinafter and the conditions of which specific to each extract have been specified in Table 1.

General Protocol:

A mass of aerial portions of loosestrife, dried beforehand, has been ground and set to macerate for 24 hours in a maceration solvent. During the entire duration of the maceration, the mixture has been maintained under stirring, at 20° C. After maceration, the plant residue has been separated from the extract by clarification and filtration.

Afterwards, the extract has been directly concentrated by vacuum evaporation, and then spray-dried and obtained in the form of a powder, with the exception of the extracts EX10MF1046 and EX13MF1046 which, before concentration, have been brought into contact with activated carbon (0.5 kg), left stirring for one hour and then separated from the activated carbon by filtration.

TABLE 1

| Reference | Mass of the | | | | | | |
|---|---|---|---|---|---|---|---|
| of the | treated | | Maceration solution | | | Extract | |
| extract | loosestrife | | Mass | | Solvent | Mass | |
| EX3MF1046 | 5 | kg | 50 | kg | Ethanol in water | 550 | g |
| | | | | | 30% (v/v) | | |
| EX10MF1046 | 5 | kg | 50 | kg | Ethanol in water | 300 | g |
| | | | | | 30% (v/v) | | |
| EX4MF1046 | 50 | g | 500 | g | Water | 3 | g |
| EX1MF1046 | 50 | g | 500 | g | Ethanol in water | 2 | g |
| | | | | | 90% (v/v) | | |
| EX2MF1046 | 50 | g | 500 | g | Ethanol in water | 4 | g |
| | | | | | 90% (v/v) | | |
| EX13MF1046 | 25 | g | 500 | g | Ethyl acetate | 300 | mg |

### Example 2: Isolation of a Compound of the Invention from a Loosestrife Extract and Characterization

1) Materials and Methods

Extract

A loosestrife (Lythrum salicaria L.) extract has been obtained by maceration of the flowering tops in a hydroalcoholic solution (ethanol 30%) for 24 hours. Afterwards, the extract has been concentrated by evaporation and then lyophilized.

Purification

A first purification step is carried out by solid-phase extraction (SPE). The chosen support is a C18 bonded silica. The extract is deposited in-solution in a hydroalcoholic solution containing 20% of methanol and 0.5% of formic acid. The elution has been carried out with the same solvent. The obtained fraction has been concentrated by evaporation and then lyophilized.

A second purification step has been carried out by semi-preparative HPLC on a Synergi Fusion (Phenomenex) column having the dimensions 250×10 mm. The elution has been ensured by a gradient of formic acid 0.5% and methanol.

Characterization

The gallic acid has been identified by comparison of the physicochemical characteristics (retention, ultraviolet absorption spectrum and mass spectrum) of the compound present in the extract with those of a commercial sample of the gallic acid (Sigma Aldrich—reference 27645).

The other purified compounds have been characterized by Nuclear Magnetic Resonance (NMR) spectroscopy of the proton (400 MHz) and of the carbon 13 (100 MHz).

2) Results

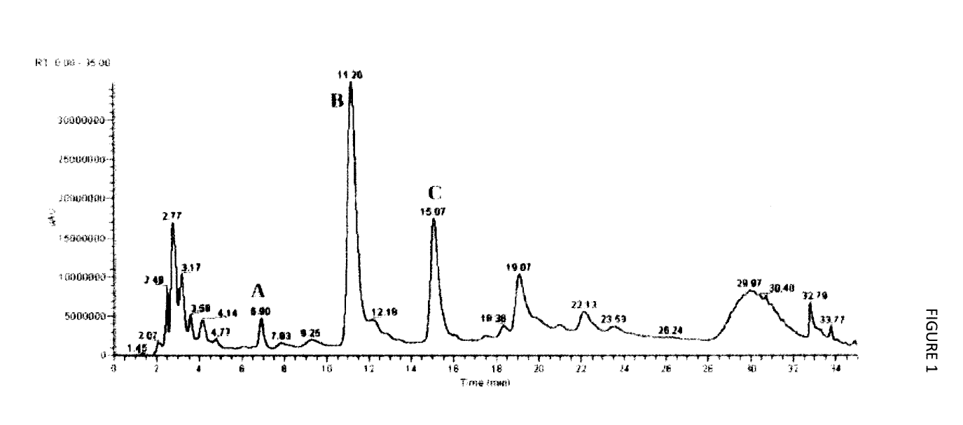

FIG. 1 represents the chromatogram of the loosestrife extract.

The compound A presents a maximum UV absorbance at 269 nm and a monoisotopic molar mass of 170 g/mol. Its retention time under the used HPLC conditions is 6.90 minutes. The commercial sample of the gallic acid presents exactly the same physicochemical characteristics. These elements allow identifying the compound A as being the gallic acid.

Tables 2 and 3 below gather together the chemical shifts of the compound B, respectively by carbon 13 NMR and by proton NMR. All these chemical shifts allow identifying the compound B as being vescalagin.

TABLE 2
Peak B

| Signal | Chemical shift (ppm) | Attribution | Characteristic |
|---|---|---|---|
| 1 | 65.15 | C6 | sugar C |
| 2 | 65.37 | C1 | sugar C |
| 3 | 68.29 | C3 | sugar C |
| 4 | 69.17 | C4 | sugar C |
| 5 | 71.03 | C5 | sugar C |
| 6 | 77.61 | C2 | sugar C |
| 7-36 | 107.26-147.30 | 30 carbons | aromatic C |
| 37 | 165.37 | C═O | ester function |
| 38 | 165.49 | C═O | ester function |
| 39 | 166.50 | C═O | ester function |
| 40 | 167.23 | C═O | ester function |
| 41 | 169.27 | C═O | ester function |

TABLE 3
Peak B

| Proton | Chemical shift δ (ppm) | Spectral data |
|---|---|---|
| H1 | 4.87 | d; 1; J = 2 Hz |
| H2 + H4 | 5.19-5.23 | m; 2H |
| H3 | 4.56 | dd; 1H; J = 0.8 et 6.8 Hz |
| H5 | 5.65 | d; 1H; J = 6.4 Hz |
| H6 | 5.08 | dd; 1H; J = 2.8 et 13.2 Hz |
| H6′ | 4.01 | d; 1H; J = 12.8 Hz |
| aromatic H | 6.61; 6.76; 6.77 | s; 1H |

Tables 4 and 5 gather together the chemical shifts of the compound C, respectively by carbon 13 NMR and by proton NMR. All these chemical shifts allow identifying the compound C as being castalagin.

TABLE 4
Peak C

| Signal | Chemical shift (ppm) | Attribution | Characteristic |
|---|---|---|---|
| 1 | 64.65 | C6 | sugar C |
| 2 | 65.62 | C1 | sugar C |
| 3 | 66.38 | C3 | sugar C |
| 4 | 68.51 | C4 | sugar C |
| 5 | 70.50 | C5 | sugar C |
| 6 | 73.36 | C2 | sugar C |
| 7-36 | 106.85-145.99 | 30 carbons | aromatic C |
| 37 | 164.04 | C═O | ester function |
| 38 | 164.98 | C═O | ester function |
| 39 | 165.99 | C═O | ester function |
| 40 | 166.55 | C═O | ester function |
| 41 | 168.58 | C═O | ester function |

TABLE 5
Peak C

| Proton | Chemical shift δ (ppm) | Spectral data |
|---|---|---|
| H1 | 5.71 | d; 1H; J = 4.40 Hz |
| H2 + H3 | 5.03-5.05 | m; 2H; J = 5.6 Hz |
| H4 | 5.25 | t; 1H; J = 7.20 Hz |
| H5 | 5.62 | dd; 1H; J = 1.60 et 8.00 Hz |
| H6 | 5.11 | dd; 1H; J = 2.40 et 12.80 Hz |
| H6′ | 4.00 | d; 1H; J = 12.80 Hz |
| aromatic H | 6.63; 6.78; 6.79 | s; 1H |

### Example 3: Evaluation of the Compounds of the Invention in Primary Cultures of Normal Human Epidermal Keratinocytes (NHEK)

1) Materials and Methods

Cells

Normal human epidermal keratinocytes (NHEK); reference BIOalternatives K341, used at the third passage.

Culture conditions: 37° C., 5% CO₂

Culture medium: keratinocytesSFM complemented with an epidermal growth factor (EGF) 0.25 ng/ml, a pituitary extract (PE) 25 μg/ml and gentamycin 25 pig/ml.

Experiment medium: keratinocytesSFM complemented with gentamycin 25 μg/ml.

Tested Compounds

After the prior evaluation of cytotoxicity, the gallic acid, the vescalagin, the castalagin and the salicarininA compounds are tested at the following concentrations:

Gallic acid, 0.0033; 0.01 and 0.03 mg/ml

Vescalagin, 0.0011; 0.003 and 0.01 mg/ml

Castalagin, 0.0011; 0.003 and 0.01 mg/ml

Salicarinin A, 0.0011; 0.003 and 0.01 mg/ml

Immunolabeling In Situ

The keratinocytes have been sowed and cultured to confluence in 96-well plates and then, the medium has been replaced by an experiment medium containing, or not (control test), the tested extract or the reference (CaCl₂ at 1.5 mM) and the cells have been incubated for 72 hours. All experimental conditions have been realized with n=3, for each marker.

After incubation, the test medium has been withdrawn and the cells have been rinsed, fixed and permeabilized. The cells have been marked with the primary antibodies directed against the considered proteins (TGK). These antibodies have been revealed by a secondary antibody coupled to a fluorochrome (GAMAlexa 488). In parallel, the nucleuses of the cells have been stained by the Hoechst 33258 (bisbenzimide). The acquisition of the images has been carried out with a high-resolution imaging system, INCell Analyzer™ 1000 (GE Healthcare). For each well, 5 captures of digitized images have been performed. The labelings have been quantified by measurement of the fluorescence intensity of the proteins divided by the number of nucleuses identified by the Hoechst (integration of the digital data by the software Developer Toolbow 1.5, GE Healthcare).

2) Results

Table 6 illustrates the expression level of the TGK via the fluorescent labeling of the NHEK for different concentrations of vescalagin, castalagin and salicarinin A.

TABLE 6

| | | | Basic data | | | | | | | | |
|---|---|---|---|---|---|---|---|---|---|---|---|
| | | | TGK | | | | | | | | |
| | | | Fluorescence | | | | | | | | |
| Treatment | | | intensity/ | | | | | | Normalized data | | |
| Tested | | | Number of | Average | csm | % | [symbol] m | | % | [symbol] m | |
| compounds | Concentration | | cells (UA) | (UA) | (UA) | Control test | % | p [symbol] | Stimulation | % | p [symbol] |
| Control test | — | | 3065 | 6167 | 1734 | 100 | 28 | * | 0 | 28 | * |
| | | | 6330 | | | | | | | | |
| | | | 9085 | | | | | | | | |
| CaCl₂ | 1.5 | mM | 24066 | 29160 | 5375 | 473 | 87 | * | 373 | [symbol] 7 | * |
| | | | 30013 | | | | | | | | |
| | | | 23527 | | | | | | | | |
| Vescalagin | 0.0011 | mg/ml | 22033 | 17722 | 2250 | 287 | 36 | * | 187 | 36 | * |
| | | | 16688 | | | | | | | | |
| | | | 14446 | | | | | | | | |
| | 0.003 | mg/ml | 32859 | 42460 | 6326 | 589 | 103 | ** | 589 | 103 | ** |
| | | | 40124 | | | | | | | | |
| | | | 54396 | | | | | | | | |
| | 0.01 | mg/ml | 27714 | 43284 | 8449 | 702 | 137 | * | 602 | 137 | * |
| | | | 45383 | | | | | | | | |
| | | | 98755 | | | | | | | | |
| Castalagin | 0.0011 | mg/ml | 21020 | 16135 | 2635 | 202 | 44 | * | 162 | 44 | * |
| | | | 15623 | | | | | | | | |
| | | | 11760 | | | | | | | | |
| | 0.003 | mg/ml | 30752 | 26867 | 4995 | 436 | 81 | * | 330 | [symbol] 1 | * |
| | | | 20672 | | | | | | | | |
| | | | 23177 | | | | | | | | |
| | 0.01 | mg/ml | 59547 | 44055 | 112 [symbol] | 714 | 183 | * | 614 | 183 | * |
| | | | 22164 | | | | | | | | |
| | | | 50453 | | | | | | | | |
| Salicarinin A | 0.0011 | mg/ml | 7348 | 11451 | 4484 | 188 | 73 | ns | 86 | 73 | ns |
| | | | 20400 | | | | | | | | |
| | | | 8597 | | | | | | | | |
| | 0.003 | mg/ml | 10264 | 12230 | 1025 | 198 | 26 | ns | 98 | 26 | ns |
| | | | 15462 | | | | | | | | |
| | | | 10987 | | | | | | | | |
| | 0.01 | mg/ml | 41338 | 38273 | 3288 | 621 | 53 | *** | 521 | 53 | *** |
| | | | 41770 | | | | | | | | |
| | | | 31701 | | | | | | | | |

[symbol]

indicates data missing or illegible when filed

Table 7 illustrates the expression level of the TGK via the fluorescent labeling of the NHEK for different concentrations of the gallic acid.

TABLE 7

| | | | Basic data | | | | | | | | |
|---|---|---|---|---|---|---|---|---|---|---|---|
| | | | TGK | | | | | | | | |
| | | | Fluorescence | | | | | | | | |
| Treatment | | | [symbol] ntensity/ | | | | | | Normalized data | | |
| Tested | | | Number of | Average | [symbol] m | % | [symbol] m | | % | [symbol] m | |
| compounds | Concentration | | cells (UA) | (UA) | (UA) | Control test | (%) | p [symbol] | Stimulation | (%) | p [symbol] |
| Control test | [symbol] | | 4265 | 6211 | 1475 | 100 | 24 | * | 0 | 24 | * |
| | | | 9105 | | | | | | | | |
| | | | 5262 | | | | | | | | |
| Ca [symbol] | 1.5 | mM | 22987 | 20404 | 2911 | 425 | 47 | ** | 325 | 47 | ** |
| | | | 24146 | | | | | | | | |
| | | | 32180 | | | | | | | | |
| Gallic acid | 0.0003 | mg/ml | 18328 | 16260 | 853 | 285 | 15 | ** | 165 | 15 | ** |
| | | | 15263 | | | | | | | | |
| | | | 15730 | | | | | | | | |
| | 0.01 | mg/ml | 47924 | 42738 | 3441 | 688 | 55 | *** | 588 | 55 | *** |
| | | | 36228 | | | | | | | | |
| | | | 44065 | | | | | | | | |
| | 0.03 | mg/ml | 73870 | 60518 | 8554 | 1110 | 138 | ** | 1019 | 138 | ** |
| | | | 53013 | | | | | | | | |
| | | | 81670 | | | | | | | | |

[symbol]

indicates data missing or illegible when filed

In Tables 6 and 7, the statistical significance threshold is as follows:

Ns: >0.05, Not significant

*: from 0.01 to 0.05, Significant

**: from 0.001 to 0.01, Very significant

***: <0.001, Extremely significant

These results show that the gallic acid, the vescalagin, the castalagin and the salicarinin A induce the TGK expression in the NHEK cultures in a dose-dependent fashion. The activity is higher than that of the calcium chloride reference, as by the concentration of 10 μg/ml.

### Example 4: Evaluation of Compounds of the Invention on Reconstructed Skins

1) Materials and Methods

Reconstructed Skins (RS)

Human skins reconstructed from a culture of keratinocytes sowed on a collagen lattice containing fibroblasts.

The keratinocytes are cultured at 37° C., 5% CO₂, at the air-liquid interface in one of the following media:

Complete culture medium: Epilife and supplements (without IGF-1)+CaCL₂ 1.5 mM+bovine insulin 5 μg/ml+vitamin C 50 μg/ml+KGF (growth factor of the keratinocytes) 3 ng/ml, or

Depleted culture medium: Epilife and supplements (without IGF-1 and without hydrocortisone)+CaCl₂ 1.5 mM+vitamin C (50 μg/ml).

Tested Compounds

Loosestrife extract obtained in Example 2 tested at 30 μg/ml and 60 μg/ml

Vescalagin tested at 30 μg/ml and 60 μg/ml

Description of the Test

The following cultures have been realized:
Untreated reconstructed skins,
Skins reconstructed in a depleted medium,
Skins reconstructed in a depleted medium, each treated by a compound tested hereinabove, at a concentration of 30 μg/ml or 60 μg/ml.

The reconstructed skins have been placed during the passage at the air-liquid interface (D0) in a 6-well plate in the depleted culture medium containing, or not (deficient control condition), the tested compounds. Afterwards, the reconstructed skins have been cultured for 9 days with the renewal of the culture medium containing, or not, the tested compounds every 2 to 3 days from D0 to D5, and then every day between D5 and D9. In parallel, a culture of the reconstructed skins in a complete medium has been realized, as a control test.

All experimental conditions have been realized with n=4.

The evaluation of the tested compound has been performed:
By histological analysis, by the histological observation of the epidermal thickness after HES (Hematoxylin-Eosin-Saffron) staining,
By RT-qPCR, by analysis of the expression of 16 markers (2 of which are reference genes) selected for their importance in cutaneous ageing, on the mRNAs extracted from the reconstructed skins for each treatment, and
By quantifying the release of MMP-1, MMP-3 and procollagen I in the suspensions of the cultures on D9, by ELISA assay, using ELISA assay kits used according to the instructions of the supplier.

2) Results

HES Analysis

Reconstructed Skins in a Complete Medium, Untreated:

They present a normal histology with the presence of the structures characteristic of a reconstructed skin:

A dermal compartment composed of fibroblasts in a collagen matrix

An epidermal compartment with the different layers of cells:

A basal layer (a layer of cells laid in palisade)

A spiny layer (4 to 5 layers of cells)

A granular layer (a layer of cells presenting grains of keratohyalin), and

A horny layer (enucleated dead cells).

Reconstructed Skins in a Depleted Medium, Untreated:

They present a modified histology in comparison with the untreated reconstructed skins with:

General destructuration of the epidermis with:

Loss of the basal layer

A less significant granular layer (decrease of the number of grains of keratohyalin)

Thick horny layer and parakeratosis

Loss of the epidermis/dermis cohesion represented by the <<slip>> or the detachment of the epidermis relative to the dermis

General aspect of the reconstructed skins: A larger thickness and a smaller length (shriveled).

Reconstructed Skins in a Depleted Medium. Treated with the Loosestrife Extract at 30 μg/Ml and 60 μg/Ml:

They present an improvement of their histology with:

Recovery of some cellular layers:

A basal layer (a layer of cells laid in palisade)

A more significant granular layer

Recovery of the epidermis/dermis cohesion

Recovery of the general dimensions observed on the untreated reconstructed skins.

Reconstructed Skins in a Depleted Medium. Treated with Vescalagin at 30 μg/Ml and 60 μg/Ml:

They present an improvement of their histology with:

Recovery of some cellular layers:

A basal layer (a layer of cells laid in palisade)

A more significant granular layer

Partial recovery of the epidermis/dermis cohesion

Recovery of the general dimensions observed on the untreated reconstructed skins.

RT-qPCR

The results are presented in Tables 8 (loosestrife extract) and 9 (vescalagin) below.

In these tables, the grayed cells of the columns [% Control test Average HK] correspond to an inhibitor or stimulating effect of the loosestrife extract or of the vescalagin.

The profiles of the expression of the selected genes are very different between the reconstructed skins cultured in a complete medium and those cultured in a depleted medium. The reconstructed skins in a depleted medium present a very high increase of the expression of the markers of the degradation of the matrix (MMP1, MMP2, TFPI2), of the markers of inflammation (PTGS2, IL1B), and of the markers of the differentiation of the keratinocytes (FLG, TGM1, CDSN). In parallel, a very high decrease of the expression of the marker of the cell cycle MKI67, of the expression of the marker of the extracellular matrix COLIA1 and of the makers of the basal keratinocytes, KRT5 and KRT14, is observed. These results are expected and validate the experiment.

The loosestrife extract, tested systemically at 30 μg/ml and 60 μg/ml in a deficient medium has allowed compensating, in a dose-dependent fashion, the effect of the deficient medium in particular with:
an effect at the level of the markers of the cells of the basal layer with an increase of the expression of the epidermal differentiation marker KRT5 (KRT14 to a lesser extent) and an increase of the marker of the cell cycle MK167. These modifications are in correlation with the histological observations which show the recovery of a distinct basal layer subsequently to the treatment;
an effect at the level of the inflammation markers with a decrease of the expression of PTGS2 and IL1B;
an effect at the level of the more tardive markers of the differentiation of the keratinocytes with a decrease of the expression of TGM1 and CDSN. These results are in correlation with the histological observations which show a better terminal differentiation of the keratinocytes (the granular layer and the horny layer becoming closer to the <<control>> reconstructed skins);
an effect at the level of the markers involved in the degradation of the extracellular matrix with a decrease of the expression of the genes MMP1, MMP3 and TFPI2. In parallel, an increase of the expression of the marker of the extracellular matrix COLIA1 is observed.

The vescalagin, tested systemically at 30 μg/ml and 60 μg/ml in a deficient medium has allowed partially compensating, in a dose-dependent fashion, the effect of the deficient medium in particular with:
an effect at the level of the markers of the cells of the basal layer with an increase of the marker of the cell cycle MKI67. These modifications are in correlation with the histological observations which show the recovery of a distinct basal layer subsequently to the treatment;
an effect at the level of the inflammation markers with a decrease of the expression of IL1B and PTGS2 to a lesser extent;
a slight effect at the level of the more tardive markers of the differentiation of the keratinocytes with a decrease of the expression of TGM1 and CDSN. These results are in correlation with the histological observations which show a better terminal differentiation of the keratinocytes (the granular layer and the horny layer becoming closer to the (<control, reconstructed skins);
an effect at the level of the markers involved in the degradation of the extracellular matrix with a decrease of the expression of the genes MMP1, MMP3 and TFPI2. In parallel, an increase of the expression of the marker of the extracellular matrix COLIA1 is observed.

ELISA Assays of MMP-1, MMP-3 and Procollagen I

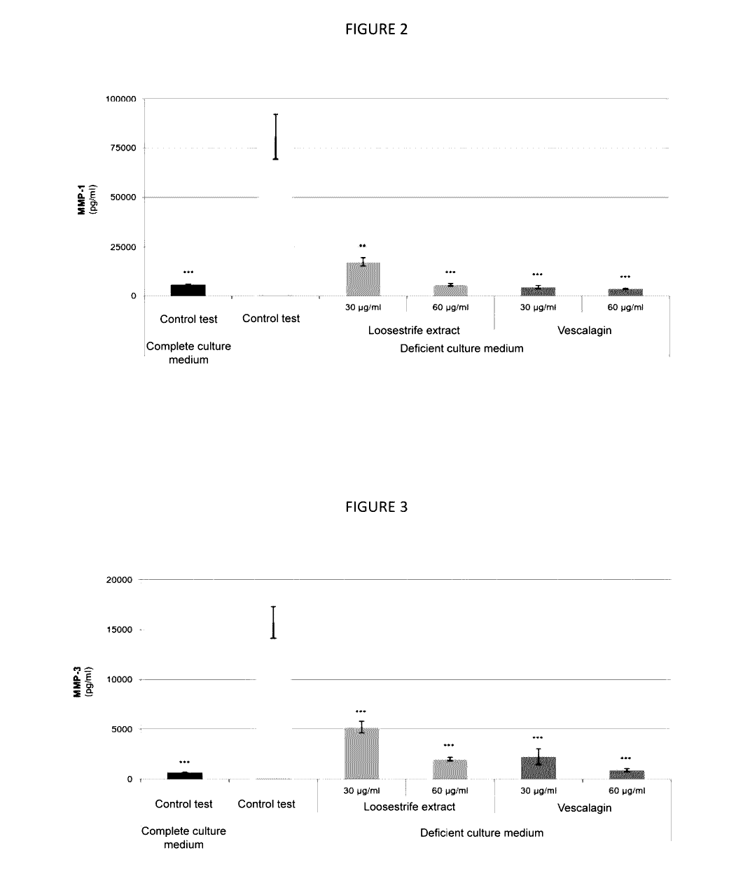

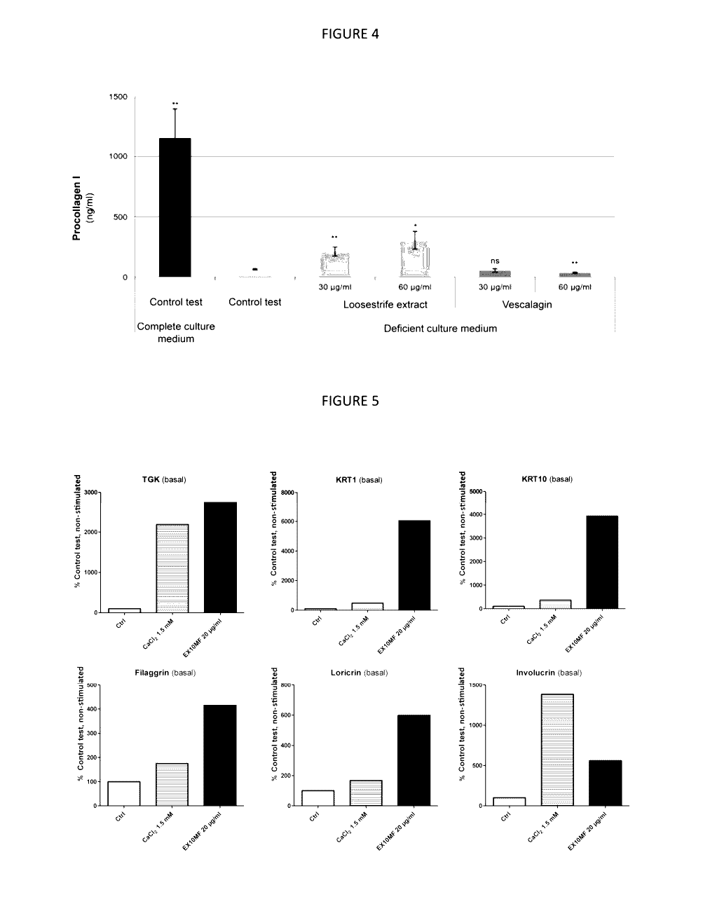

The results of the assays in the culture suspensions have been presented in FIG. 2 (MMP-1), FIG. 3 (MMP-3) and FIG. 4 (procollagen I).

As regards the reconstructed skins in a depleted culture medium, it is observed:
A very high increase of the release of proteases involved in the degradation of the extracellular matrix, MMP-1 and MMP-3, and
A clear decrease of the release of a protein of the extracellular matrix, the procollagen I.

The systemic treatment, by the loosestrife extract at 30 μg/ml and 60 μg/ml, of the reconstructed skins in a depleted culture medium induces a major and significant decrease of the release of MMP-1 and MMP-3. In parallel, it significantly stimulates the release of procollagen I.

The systemic treatment, by vescalagin at 30 μg/ml and 60 μg/ml, of the reconstructed skins in a depleted culture medium induces a major and significant decrease of the release of MMP-1 and MMP-3. No modification has been observed on the release of procollagen I.

It follows from this example that the loosestrife extract and the vescalagin have protected the reconstructed skins from ageing induced by the depleted (deficient) culture medium.

### Example 5: Evaluation of Loosestrife Extracts in Primary Cultures of Normal Human Epidermal Keratinocytes (NHEK)

1) Materials and Methods

Cells

Normal human epidermal keratinocytes (NHEK); reference BIOalternatives K341, used at the third passage.

Culture conditions: 37° C., 5% CO₂

Culture medium: keratinocytesSFM complemented with an epidermal growth factor (EGF) 0.25 ng/ml, a pituitary extract (PE) 25 μg/ml and gentamycin 25 μg/ml.

Experiment medium: keratinocytesSFM complemented with gentamycin 25 μg/ml.

Tested Extracts

In the initial step of selecting an extract, and after the prior evaluation of cytotoxicity, the extracts EX1MF1046, EX2MF1046, EX3MF1046, EX4MF1046 and EX13MF1046 of Example 1, are tested at the following concentrations:

EX1MF1046, 0.005 mg/ml

EX2MF1046, 0.003 mg/ml

EX3MF1046, 0.005 mg/ml

EX4MF1046, 0.005 mg/ml

EX13MF1046, 0.005 mg/ml

The step hereinabove results in the retention of the extracts EX3MF1046 and EX10MF1046 which corresponds to the filtered extract EX3MF1046, for the following experiments. They are evaluated at the following concentrations:

EX3MF1046, between 0.0016 mg/ml and 0.02 mg/ml; non-cytotoxic concentration.

EX10MF1046, 0.02 mg/ml

Immunolabeling In Situ

The keratinocytes have been sowed and cultured to confluence in 96-well plates and then, the medium has been replaced by an experiment medium containing, or not (control test), the tested extract or the reference (CaCl₂ at 1.5 mM) and the cells have been incubated for 72 hours. All experimental conditions have been realized with n=3, for each marker.

After incubation, the test medium has been withdrawn and the cells have been rinsed, fixed and permeabilized. The cells have been marked with the primary antibodies directed against the considered proteins (TGK, filaggrin and KRT10)¹. These antibodies have been revealed by a secondary antibody coupled to a fluorochrome (GAMAlexa 488). In parallel, the nucleuses of the cells have been stained by the Hoechst 33258 (bisbenzimide). The acquisition of the images has been carried out with a high-resolution imaging system, INCell Analyzer™ (000 (GE Healthcare). For each well, 5 captures of digitized images have been performed. The labelings have been quantified by measurement of the fluorescence intensity of the proteins divided by the number of nucleuses identified by the Hoechst (integration of the digital data by the software Developer Toolbow 1.5, GE Healthcare).

Screening of the extracts has been carried out only on the TGK parameter (Table 10).

Analysis Des Transcripts, RT-qPCR

The keratinocytes have been sowed and cultured to confluence in 24-well plates and then the medium has been replaced by an experiment medium containing, or not (control test), the experimented compound or the reference (CaCl₂ at 1.5 mM) and the cells have been incubated for 48 hours. All experimental conditions have been realized with n=3, for each marker.

After incubation, the experiment medium has been withdrawn and the cells have been rinsed and frozen down to −80° C.

The expression of the markers has been evaluated by RT-qPCR on the messenger RNAs (mRNA) extracted from the cellular monolayers of each treatment (the replicates have been pooled before the extraction of the RNA). The RNA extraction steps by reverse transcription, the PCR primers and conditions have been described before.

2) Results

Screening

The results are presented in Table 10 below which illustrates the expression level of the TGK via the fluorescent labeling of the NHEK, the fluorescence intensity of the NHEK being proportional to the TGK expression.

TABLE 10

| | Tested compound | | | | | |
|---|---|---|---|---|---|---|
| | Control | EX1MF1046 | EX2MF1046 | EX3MF1046 | EX4MF1046 | EX13MF1046 |
| | test | 0.005 mg/ml | 0.003 mg/ml | 0.005 mg/ml | 0.005 mg/ml | 0.01 mg/ml |
| TGK expression | / | ++ | ++ | ++++ | ++ | / |
| rate | | | | | | |

The extract EX3MF1046 turns out to be the most active under these conditions.

Influence of the Extract EX3MF1046 at Different Concentrations, on the TGK Expression

Table 11 illustrates the expression level of the TGK via the fluorescent labeling of the NHEK for different concentrations of the extract EX3MF1046.

TABLE 11

| Treatment | | | | | | |
|---|---|---|---|---|---|---|
| Tested | | | TGK expression^{a)} | | Control test | |
| compound | Concentration | | Average | ESM | % | ESM |
| Control test | — | | 22042 | 3399 | 100 | 15 |
| CaCl₂ | 1.5 | mM | 69610 | 681 | 316 | 3 |
| EX3MF1046 | 0.0016 | mg/ml | 41326 | 3696 | 187 | 17 |
| | 0.005 | mg/ml | 133494 | 4753 | 606 | 22 |
| | 0.01 | mg/ml | 201936 | 7097 | 916 | 32 |
| | 0.02 | mg/ml | 320616 | 6643 | 1455 | 30 |

The TGK expression is given as the Fluorescence intensity/Number of cells (UA)

These results show that the extract EX3MF1046 induces the TGK expression in the NHEK cultures in a dose-dependent fashion. The activity is higher than that of the calcium chloride reference, as by the concentration of 5 μg/ml; the maximum effect which is 4 times higher than that of CaCl₂ is observed at the non-cytotoxic concentration of 20 μg/ml.

Influence of the Extract EX10MF1046, on the TGK Expression, the Filaggrin Expression and the KRT10 Expression

The same methodology employed for evaluating the effect of an extract on the TGK expression has been applied for the filaggrin and KRT10 markers.

Table 12 presents an evaluation of the expression level of the three markers hereinabove via the fluorescent labeling of the NHEK.

TABLE 12

| Treatment | | | | |
|---|---|---|---|---|
| Tested | | TKG | Filaggrin | KRT10 |
| compound | Concentration | expression | expression | expression |
| Control test | — | +/− | − | + |
| CaCl₂ | 1.5 mM | +++ | +++ | +++ |
| EX10MF1046 | 20 μg/ml | ++++ | ++ | ++++ |

It is observed that the filtration of the extract EX3MF1046 maintains the pro-differentiating effect of the extract.

In this experiment, the inductor effect of the TGK is revealed and a very significant over-expression of the protein KRT10 is observed. In contrast with the calcium which focuses the effect on few cells by transforming them into very large differentiated cells (comeocytes), the EX10MF1046 induces a very significant over-expression of KRT10 in numerous cells but does not significantly increase the size of the cells. Hence, the action mechanisms of the calcium and of the EX10MF1046 are different and may by additive or synergistic. This is confirmed by the analysis of the fiaggrin, which increases (more moderately) subsequently to the treatment by EX10MF1046, but without inducing the characteristic production of filaggrin granules in the cells in response to the calcium.

Influence of the Extract EX10MF1046, on the Expression of Other Markers of the Epidermal Differentiation

The analysis has been broadened to other protein markers of the epidermal differentiation, namely, the loricin, the involucrin and the keratin 1 (KRT1).

The influence of EX10MF1046 at 20 μg/ml is studied on the expression of the transcripts of the different markers hereinabove by the cultures of keratinocytes in the basal state. The treatments have lasted 48 h, the analysis has been carried out by RT-qPCR. The results are given in % relative to the untreated control tests after randomization of the relative expressions with reference to the housekeeping gene GAPDH. They are represented in FIG. 5.

The RT-qPCR analyses show a very clear over-expression of all the differentiation markers in the presence of the extract; the stimulations have been significant (stimulation with a factor of 4 several times depending on the markers) and all have been higher than those obtained with calcium, with the exception of the involucrin marker.

### Example 6: Evaluation of Loosestrife Extracts in Reconstructed Human Epidermises (RHE)

1) Materials and Methods

Reconstructed Human Epidermises

RHE produced from keratinocytes of the human prepuce and widely described in the BIOalternatives publication¹⁻⁴.

The RHE have been cultured at 37° C., 5% CO₂, at the air-liquid interface, in a medium containing Epilife+calcium 1.5 mM and supplements, without the vitamin C. The reference for the studies of the pro-differentiating effects is the vitamin C (50 μg/ml).

Tested Extracts

The extract EX10MF1046 of Example 1 has been tested at 20 μg/ml, in the RHE culture medium.

Immunolabeling In Situ

The RHE are placed at the air-liquid interface (D0) and cultured for 7 days in the presence or absence (control test) of the extract EX10MF1046 or the vitamin C (reference).

At the end of the incubation, the epidermises have been rinsed and then fixed in a solution of formaldehyde. The fixed tissues have been dehydrated by successive and increasing ethanol baths and then included in paraffin <<Paraplast>>. Cross-sections have been realized to the microtome scale (thickness 5 μm) and then maintained at ambient temperature until the realization of the labelings.

The sections have been dewaxed and then the antigenic sites have been unmasked in a citrate buffer solution at pH6 (DAKO, S1700). After washing in PBS-T, the sections have been incubated with a solution of hydrogen peroxide at 3% for 5 minutes. After washing, the sections have been incubated afterwards at ambient temperature for 1 hour with the primary antibody (anti-TGK, SC-25786; anti-KRT10, SC-23877; anti-filaggrin, SC-66192; all from Santa-Cruz Biotech). After washing, the labeling has been revealed with a streptavidin-peroxidase detection kit (DAKO, ref. K0690) coupled with the AEC chromogen (DAKO, K346111). After counterstaining with hematoxylin and washing in ultrapure water, the sections have been mounted between slide and coverslip in an aqueous Glycergel medium (DAKO, C056330). The sections have been observed using a microscope NIKON E400. The digital images have been recoded with a camera KOKON DS-Ril and the software NIS-Elements 3.10.

2) Results

The passage at the air-liquid interface results, under the optimum epidermal reconstruction conditions, in a very significant differentiation which is very difficult to over-stimulate. Therefore, it is appropriate to settle in non-optimum or in slightly deficient conditions in order to see the effect of the products stimulating the differentiation.

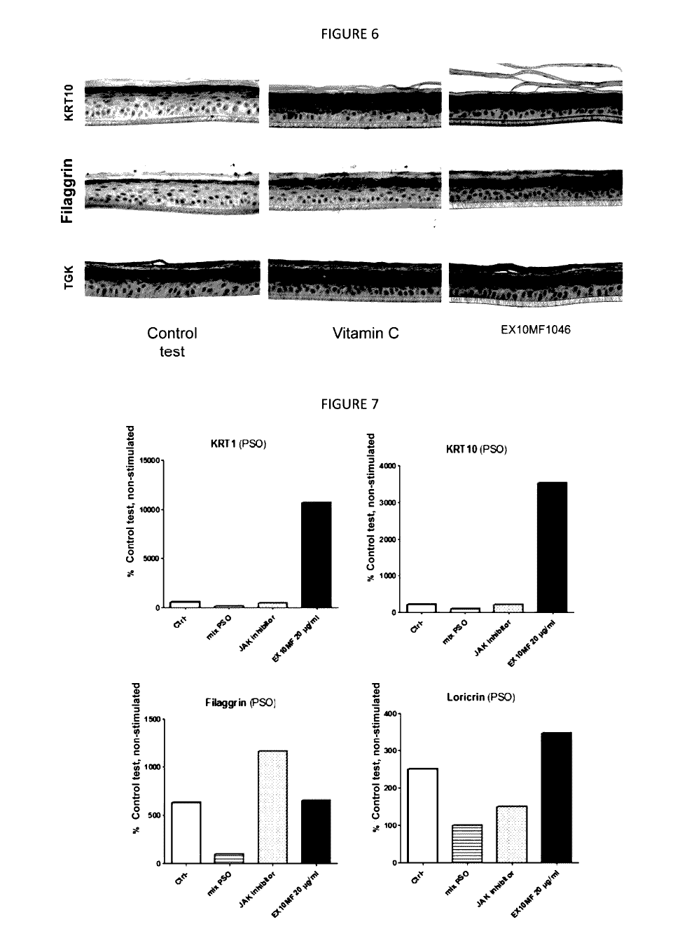

The obtained digital images are presented in FIG. 6.

In these experimental conditions presenting a deficiency (vitamin C), the extract EX10MF1046 shows a clear compensation and a significant stimulation of the expression of the KRT10, filaggrin and TGK.

### Example 7: Protective Effects of Loosestrife Extracts on Cutaneous Lesions Induced by the Inflammation (Psoriasis, Dermatitises) of the Epidermis; Evaluation in Reconstructed Epidermises (RHE)

1) Materials and Methods

Reconstructed Cells and Epidermises

The NHEK have been prepared as in Example 5.

The RHE have been prepared as in Example 6 (complete medium including vitamin C).

Inflammatory Mixture, Reference

The inflammatory mixture (mix cytokines, M3) is constituted by interleukin-17 (IL-17), oncostatin M (OSM) and tumor-necrosis factor alpha (TNFα), all provided by R&D Systems, each at the final concentration of 3 ng/ml.

The reference is <<JAK inhibitor 1>> (Calbiochem CAS 457081-03-7) at the final concentration of 10 μM.

Tested Extract

The extract EX10MF1046 of Example 1 is tested at 20 μg/ml, in the culture medium of the RHE.

Analysis Des Transcripts, RT-qPCR

The NHEK have been pre-cultured as in Example 5 and then treated for 24 h with the extract EX10MF1046 (20 μg/ml) or the JAK inhibitor (10 μM). Afterwards, the cytokines mixture has been added to the media containing, or not, the extract and the cultures have been pursed for 24 h.

At the end of the incubation, the cellular monolayers are rinsed and then extracted in a lysis buffer; the following protocols and analyses are the same as those of Example 5.

The results are given in % relative to the untreated control tests, they are represented in FIG. 7.

Immunolabeling In Situ

The RHE have been placed at the air-liquid interface (D0) and cultured until D11. Then, the RHE have been treated systemically with the extract EX10MF1046 (20 μg/ml) or the JAK inhibitor (10 μM) for 7 h. The cytokines mixture has been added to the media containing, or not, the products and the cultures have been pursued for 48 h.

The treatment of the tissues, the labelings and the analysis have been carried out as in Example 6. Digital images of the observed sections have been recorded.

The NHEK or the RHE have been treated by the extract EX10MF1046 or the reference, and then by a cytokine mixture constituted by at least one cytokine activating the Jak/Stat pathway, namely OSM or IL-22 (STAT-3/1 activators); an activator of the NF-kB pathway, namely IL- or TNF-α and a third cytokine may be IL-17 (CREBP activator) orienting towards a psoriasis-type response or IL-4/IL-13 (STAT-6 activator), orienting towards the atopic dermatitis⁵⁻⁷. Note that all these cytokines are also substantially activators of the MAKPkinases (mitogen-activated protein kinases) pathways within the keratinocyte. In these mixtures, the STAT-3/1 inducting cytokines (OSM, IL-22)^{2-3,7} are crucial in the cutaneous pathophysiology, they are responsible of the effect on the cutaneous differentiation and the morphological modifications observed in these pathologies. The other cytokines, in particular IL-17, TNF-α and IL-1⁵⁻⁷ are more involved, and in a highly synergistic manner, in the induction of the innate immunity (antimicrobial peptides) and in the production of chemokines responsible of the maintenance and amplification of the inflammatory loop responsible of these chronic pathologies⁵⁻⁷.

This chosen mixture contains the OSM as STAT-3/1 inducer, associated to TNF-a (NF-kB, MAPkinaes) and to IL-17 (for giving the <<psoriasis>> orientation), Th17. This mixture has a highly synergistic inflammatory effect which therefore mimics the cutaneous inflammatory pathology at the level of the epidermis and in particular psoriasis. The replacement of IL-17 (cytokine Th17) by one or several cytokine(s) Th2 (IL-4/IL-13) would result in a cutaneous phenotype closer to the atopic dermatitis⁶. In both cases, the abnormal differentiation of the epidermis is remarkable, with a loss of the terminal differentiation markers, hyperplasia (etc).

The reference is the <<JAK inhibitor 1|>, a great inhibitor of the Jak-Stat pathway and tested at high concentration.

ELISA Assays

At 48 h, the culture media of the RHE have been collected and frozen down to −80° C. The IL-8 content has been assayed using a specific ELISA kit (R&D Systems DY208), according to the protocol recommended by the supplier.

2) Results

The influence of the extract EX10MF1046 at 20 μg/ml is studied on the expression of the transcripts of the markers KRT1, KRT10, filaggrin and loricrin, by the keratinocytes cultures stimulated (or not (Ctrl-)) by a mixture of cytokines (IL-17, OSM, TNFα at 3 ng/ml, mix PSO). The treatments have lasted for 48 h in all, the analysis has been carried out by RT-qPCR.

The results are represented in FIG. 7.

They show that the extract EX10MF1046 protects the epidermis from the effects of the inflammation by restoring the expression of the tardive differentiation markers, filaggrin and loricrin, and that this extract induces a significant over-expression of the keratins KRT1 and KRT10 under these conditions. Hence, the extract EX10MF1046 potentially protects from differentiation defects of the epidermis induced by the inflammation.

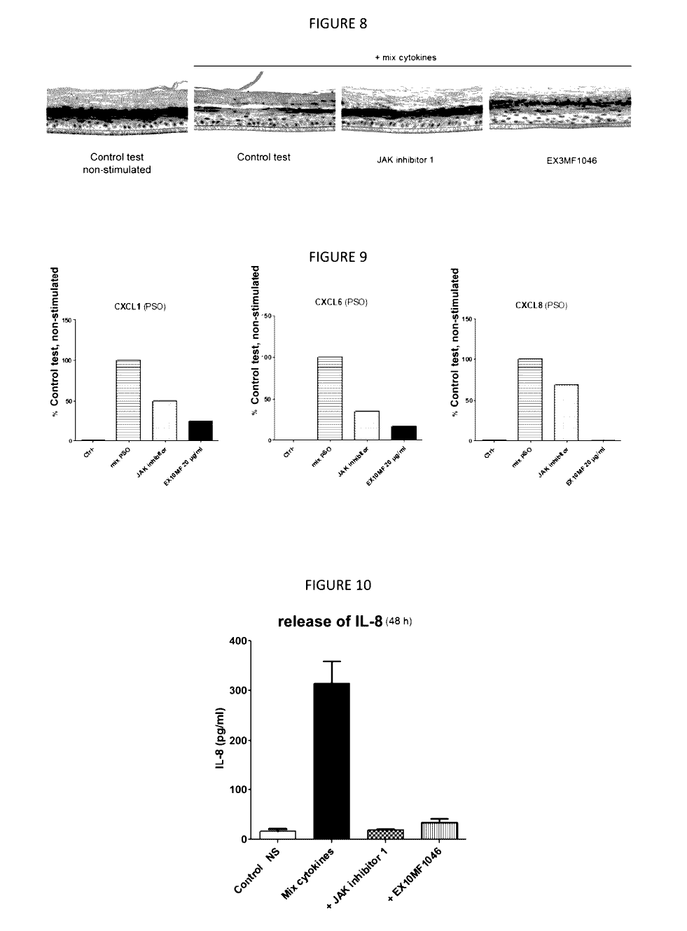

The KRT10 expression by reconstructed epidermises (RHE analyzed on D13) stimulated by the mixture of cytokines IL-17, OSM, TNFα is observed in the digital images represented in FIG. 8.

This FIG. 8 shows a partial but clear restoration of the expression of the KRT10 protein by the treatment with the extract EX10MF1046 in the RHE treated by the pro-inflammatory mixture.

FIG. 9 shows the inhibitor effect of the extract EX10MF1046 (at 20 μg/ml) on the expression of chemokines induced by the inflammation of the keratinocytes. This effect results from the interruption of the inflammatory loop by preventing the recruitment of leukocytes at the level of the lesioned skin. This effect adds to the effects on the differentiation (FIG. 7) to contribute to the return to a normal homeostasis of the skin (resolution of the inflammation).

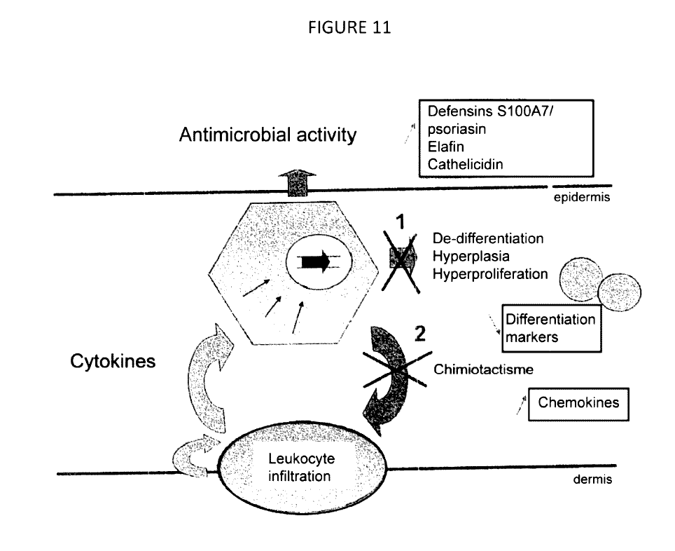

On the other hand, as has been seen at the level of the transcripts (FIG. 9), EX10MF1046 inhibits very significantly the expression/release of chemokines and in particular the IL-8 at the level of the protein (FIG. 10), thereby confirming its potential inhibitor effect on the recruitment of leukocytes (polynuclear in the case of the IL-8) at the level of the cutaneous lesions (besides the positive effect on these lesions themselves). These effects 1) protection of the cutaneous lesions and 2) limitation of the leukocyte infiltration in the skin have been summarized in FIG. 11 which schematically represents a chronic inflammation loop of the skin (psoriasis . . . ).

Hence, EX10MF1046 protects from the pernicious effects (cutaneous lesions) of the cytokines produced by the leukocytes (1), while blocking the recruitment of leukocyte infiltrates and therefore breaking the vicious circle of the chronic inflammation (2).

### Example 8: Effects of Loosestrife Extracts in Hair Reinforcement

1) Materials and Methods

Culture of Human Hair Follicles In Vitro

The hair bulbs have been micro-dissected from a lifting and placed as they are isolated in a culture medium (complemented William medium; Invitrogen) containing, or not (control test), the tested extract and then incubated for 72 hours. For each condition, an excess of bulbs has been prepared in order to reach the targeted value of 6 hairs/condition selected ultimately.

At the end of the incubation, the hairs have been dry frozen in liquid nitrogen and stored at −80° C. until the RNA extraction.

Tested Extract

The extract EX10MF1046 of Example 1 is tested at 20 μg/ml, in the culture medium of the RHE.

Analysis Des Transcripts, RT-qPCR

The isolation and the quality control of the RNAs have been carried out as in Example 5.

The amplification of the RNAs and the synthesis of the RNA biotinylated analogs (aRNA) have been carried out using the kit <<GeneChip 3′IVT Express>> (Affymetrix®)). The hybridization and the labeling have been carried out using the kit <<GeneAtlas™ hybridization, wash and stain kit for 3′IVT arrays>> (Affymetrix®). The hybridization of the fragmented aRNAs on the Affymetix® HG-U219 chip has been carried out on the hybridization station <<GeneAtlas™ fluidics station>> (Affymetrix®) for 20 hours at 45° C. All experimental and normalization procedures have been carried out according to the guidelines of the supplier.

The analysis has been performed by full transcriptome microarray on an Affymetix® HG-U219 chip.

2) Results

Keratins are major components of the hair shaft. An increase of these keratins would have substantially a positive impact on the capillary development-reinforcement.

The effects of the extract EX10MF1046 in comparison with the control test is illustrated in the following Table 13 which gives the expression of the transcripts of the differentiation markers in human hair follicles maintained alive in vitro.

TABLE 13

| | Control | Loosestrife | | | |
|---|---|---|---|---|---|
| Affy U219 | test | | Modulation | Name of the | |
| Probe Set ID | RE1 | RE2 | factor | gene | Abbreviation |
| 11723952_at | 120.1177 | 441.5676 | 3.68 | keratin 1 | KRT1 |
| 11757401_a_at | 1557.519 | 3385.916 | 2.17 | keratin 10 | KRT10 |
| 11731852_at | 54.47806 | 114.4569 | 2.10 | loricrin | LOR |
| 11738505_at | 1956.021 | 3712.088 | 1.90 | keratin | KRTAP13-1 |
| | | | | associated | |
| | | | | protein 13-1 | |
| 11757905_x_at | 295.5005 | 513.2589 | 1.74 | keratin 18 | KRT18 |
| 11759172_at | 40.7767 | 70.12949 | 1.72 | keratin | KRTAP19-4 |
| | | | | associated | |
| | | | | protein 19-4 | |
| 11759392_at | 59.00863 | 100.2564 | 1.70 | keratin | KRTAP21-2 |
| | | | | associated | |
| | | | | protein 21-2 | |
| 11731500_a_at | 264.1598 | 431.7103 | 1.63 | keratin 19 | KRT19 |
| 11738284_at | 2832.61 | 4332.157 | 1.53 | keratin | KRTAP13-2 |
| | | | | associated | |
| | | | | protein 13-2 | |

This experiment shows that the transcripts of some keratins and associated proteins have a clear tendency to increase their expression, such as KRT1, KRT10 and loricrin. This reflects again the effects on the keratinocytes. The effects are less visible than in the previous examples 1) since the follicle is less sensitive to the treatments because it is <<protected>> by the internal and external leaflets (the keratinocytes are not in direct contact with the medium) and by its own volume (the accessibility to the internal keratinocytes of the follicle) and 2) since the used microarrays method is less sensitive than the qPCR method.

1. A use of a compound, of a salt of this compound or of a derivative of this compound or of a salt of this derivative, said compound being chosen among the gallic acid, its hydrolyzable polymers in gallic acid, the hexahydroxydiphenic acid, its hydrolyzable polymers in hexahydroxydiphenic acid, the ellagic acid, its hydrolyzable polymers in ellagic acid, the gallotannins, the ellagitannins, as an active ingredient in a cosmetic composition for stimulating or repairing the barrier function of the epidermis.

2. The use of a derivative of a compound according to claim 1, wherein it has a structure which comprises at least one remainder of the hexahydroxydiphenic acid (HHDP) and a saturated aliphatic hydrocarbon chain of 6 atoms of carbon, said remainder of HHDP being attached on said chain by its carboxyl groups.

3. The use according to claim 2, wherein said hydrocarbon chain is substituted with a group chosen among the alkyl groups in C1-C6 and the cycloalkyl groups in C3-C6, possibly interrupted by at least one or several atom(s) of oxygen and possibly substituted with one or several group(s) chosen among the alkyl groups in C1-C6, the cycloalkyl groups in C3-C6, the hydroxyl group and the alkoxyl groups in C1-C6.

4. The use according to claim 1, wherein the compound is chosen among the gallic acid, the tannic acid, the 1,2,3,4,6-penta-O-galloyl-beta-D-glucopyranose, vescalagin, castalagin, casuarinin, stachyurin, salicarinin A, salicarinin B, and salicarinin C.

5. The use according to claim 1, wherein the salts of the compound are addition salts of acids chosen among the hydrochloric acid, the hydrobromic acid, the sulfuric acid, the phosphoric acid, the acetic acid, the trifluoroacetic acid, the lactic acid, the pyruvic acid, the malonic acid, the succinic acid, the glutaric acid, the fumaric acid, the tartaric acid, the maleic acid, the citric acid, the ascorbic acid, the methane- or ethane-sulfonic acid, and the camphoric acid.

6. The use according to claim 1, wherein the salts of the compound are addition salts of bases chosen among the sodium or potassium hydroxide, the trimethylamine or the tert-butylamine.

7. The use according to claim 1, wherein said compound is extracted from a plant.

8. The use according to claim 7, wherein said compound is in the form of an extract of a plant.

9. The use according to claim 7, wherein the plant is chosen in the Lythraceae family, the Fagaceae family, the Myrtaceae family, the Combretaceae family, the Elaeagnaceae family, the Melastomataceae family, the Myricaceae family or the Rosaceae family.

10. The use according to claim 9, wherein the plant is chosen among Lythrum salicaria L., Quercus sp, Castanea sp, Anogeissus leiocarpus (DC.) Guill. &Perr., Castanea sativa Mill., Corymbia citriodora, Elaeagnus rhamnoides (L.) A. Nelson, Fragaria chiloensis (L.) P. Mill., Fragaria vesca L., Lagerstroemia speciosa (L.) Pers., Melastoma candidum D. Don, Psidium guajava L., Punica granatum L., Quercus suber, Quisqualis indica, Rhodomyrtus tomentosa and Terminalia arjuna.

11. The use according to claim 1, wherein the cosmetic composition contains from 0.001 to 5% by weight of said compound relative to the total weight of the composition.

12. A non-therapeutical cosmetic use of a compound, of a salt of this compound or of a derivative of this compound or of a salt of this derivative according to claim 1, as an active ingredient in a cosmetic composition intended to be applied on a dry skin with an atopic tendency, for fighting skin ageing, for fighting itches and pruritus, for hydration and for protection from external aggressions related to pollution and stress.

13. A compound, a salt of this compound, a derivative of this compound or a salt of this derivative, said compound being chosen among the gallic acid, its hydrolyzable polymers in gallic acid, the hexahydroxydiphenic acid, its hydrolyzable polymers in hexahydroxydiphenic acid, the ellagic acid, its hydrolyzable polymers in ellagic acid, the gallotannins, the ellagitannins, for use in the treatment of lesions caused by Crohn's disease or in the treatment of lesions associated to atopic dermatitis, eczema, psoriasis, seborrheic dermatitises and acne.

14. A derivative of a compound for use in the treatment of lesions caused by Crohn's disease or in the treatment of lesions associated to atopic dermatitis, eczema, psoriasis, seborrheic dermatitises and acne, according to claim 13, wherein it has a structure which comprises at least one remainder of the hexahydroxydiphenic acid (HHDP) and a saturated aliphatic hydrocarbon chain of 6 atoms of carbon, said remainder of HHDP being attached on said chain by its carboxyl groups.

15. A pharmaceutical composition for use in the treatment of lesions caused by Crohn's disease or in the treatment of lesions associated to atopic dermatitis, eczema, psoriasis, seborrheic dermatitises and acne, characterized in that it comprises a compound, a salt of a compound, a derivative of a compound or a salt of a derivative, compatible with a pharmaceutical use, said compound being chosen among the gallic acid, its hydrolyzable polymers in gallic acid, the hexahydroxydiphenic acid, its hydrolyzable polymers in hexahydroxydiphenic acid, the ellagic acid, its hydrolyzable polymers in ellagic acid, the gallotannins, the ellagitannins.

16. The composition according to claim 15, wherein a derivative of a compound has a structure which comprises at least one remainder of the hexahydroxydiphenic acid (HHDP) and a saturated aliphatic hydrocarbon chain of 6 atoms of carbon, said remainder of HHDP being attached on said chain by its carboxyl groups.

17. The composition according to claim 15, wherein the salt of the compound is chosen among the addition salts of the hydrochloric acid, the hydrobromic acid, the sulfuric acid, the phosphoric acid, the acetic acid, the trifluoroacetic acid, the lactic acid, the pyruvic acid, the malonic acid, the succinic acid, the glutaric acid, the fumaric acid, the tartaric acid, the maleic acid, the citric acid, the ascorbic acid, the methane- or ethane-sulfonic acid, or the camphoric acid, and the addition salts of bases chosen among the sodium or potassium hydroxide, the trimethylamine or the tert-butylamine.

18. The composition according to claim 15, wherein said compound is in the form of a powder, in a form supported by absorption on a powdered organic polymer such as bentonite or talc, or in an encapsulated form.

19. The composition according to claim 15, wherein said compound is encapsulated in microspheres, liposomes, glycospheres, chylomicrons, macro-, micro-, or nanoparticles, or macro-, micro-, or nanocapsules.

20. The composition according to claim 15, wherein it contains an active substance effective in chronic inflammations, such as an active substance chosen among mucopolysaccharides, vitamins, ceramides and vegetable oils.
